(19) United States
(12) Patent Application Publication
(10) Pub. No.: US 2019/0086432 A1
(43) Pub. Date: Mar. 21, 2019

(54) SYSTEMS AND METHODS RELATED TO HUMAN NON-INVASIVE DRUG TESTING

(71) Applicants: Bao Tran, Saratoga, CA (US); Lyle Williams, Elk Ridge, UT (US)

(72) Inventors: Bao Tran, Saratoga, CA (US); Lyle Williams, Elk Ridge, UT (US)

(21) Appl. No.: 15/708,119

(22) Filed: Sep. 18, 2017

(51) Int. Cl. G01N 33/94; G01N 33/543; G01N 33/497; G01N 21/3504; G01N 27/02

(52) U.S. Cl. CPC G01N 33/948; G01N 33/54306; G01N 33/497; G01N 27/028; G01N 33/5438; G01N 21/3504; G01N 33/4972

(57) ABSTRACT

A method for detecting a substance in a subject includes providing a polymer responsive to a change in property when exposed to the substance and a material that binds the substance to Δ-9-tetrahydrocannabinol (THC) or cannabis; contacting the polymer with a fluid or a breath from a subject; and detecting a presence of the substance in the subject by detecting a change in polymer property and comparing the change to predetermined values for THC or cannabis.

### BACKGROUND

The present invention relates to systems and methods related to human non-invasive drug testing.

Before the advent of the car, being drunk was a relatively minor nuisance for society at large. Most of the time, imbibing too much alcohol did little to put strangers in harm's way. The automobile, with its speed and weight, when combined with alcohol consumption, is a major driver of traffic accidents.

In a parallel trend, marijuana is legal in many states. As states continue to loosen restrictions on marijuana safety regulators and law enforcement are struggling to figure out how to establish a legal limit for drivers, just as there is a 0.08 limit for alcohol.

Although marijuana had a less dramatic effect than alcohol on drivers, it still impairs driving performance. The drug reduced the drivers' peripheral vision giving them tunnel vision. People driving with blood concentrations of 13.1 μg/L THC, the main psychoactive ingredient in marijuana, showed increased weaving within the lane, similar to those with 0.08 breath alcohol, the threshold for impaired driving in many states.

Drinking alcohol and smoking marijuana can enhance the effect, so that drivers using both substances weaved within lanes even if their blood THC and alcohol concentrations were below the impairment thresholds for each substance alone. Alcohol, but not marijuana, increased the number of times the car actually left the lane and the speed of weaving.

Cannabis concentrations drop rapidly during the time required to collect a blood specimen in the U.S., generally within two to four hours. Oral tests using the drivers' saliva can be done roadside without a long wait but researchers found oral tests may not be a precise measure of the level of impairment. The concern is that implementing concentration-based cannabis-driving legislation will unfairly target individuals not acutely intoxicated, because residual THC can be detected in blood for up to a month of sustained abstinence in chronic frequent smokers.

### SUMMARY

A method for detecting a substance in a subject includes providing a polymer responsive to a change in property when exposed to the substance and a material that binds the substance to Δ-9-tetrahydrocannabinol (THC) or cannabis; contacting the polymer with a fluid or a breath from a subject; and detecting a presence of the substance in the subject by detecting a change in polymer property and comparing the change to predetermined values for THC or cannabis.

Advantages of the system may include one or more of the following. The system provides an easy to use, yet accurate device that can detect THC concentrations in breath or saliva. Detecting marijuana use through testing of breath or oral fluid is of interest to health professionals, law enforcement, and others concerned about personnel impairment from drug intoxication. The sampling of oral fluid for marijuana use as described herein exploits the relative ease of obtaining and testing the deposition of cannabinoid compounds in the oral mucosa following smoking of the drug. Additionally, the measurement of the marijuana-specific compound Δ-9-tetrahydrocannabinol (THC) is readily testable from oral fluid using the methods and articles described herein and is of particular interest because it remains present in the oral fluid following the onset and subsequent decline in physiological and pharmacological effects of marijuana. The methods and articles described herein improves the detection of an analyte from an unprocessed saliva sample—i.e., the assay permits detection of the THC target in a non-purified form for ingestion within the time period of impairment. The system is inexpensive and cheaper than blood testing and can quantify THC that's been recently consumed, and can detect consistent consumption of cannabis.

### BRIEF DESCRIPTION OF THE DRAWINGS

FIGS. 1 and 2 show exemplary embodiments for detecting THC.

FIGS. 3 and 4 show exemplary processes for IR detection of THC and/or alcohol.

### DESCRIPTION

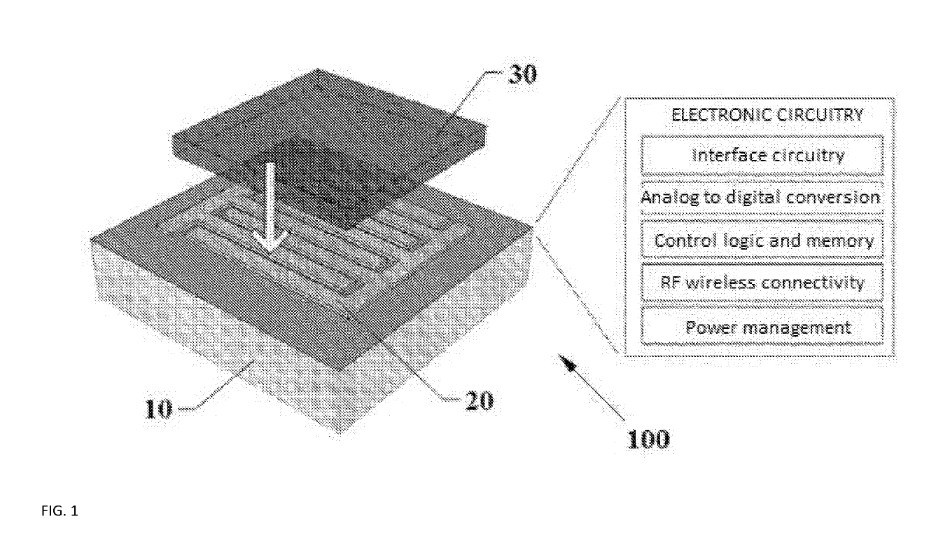

FIG. 1 shows an embodiment for detecting THC which is a highly sensitive and selective sensor. The device has a sensor material secured into a fixed position on a substrate, along with a resistive or capacitive signaling component which creates a detectable signal in response to movement of the substance deposited on the sensor. The sensitivity of the device is enhanced by using a sensor material which undergoes a dramatic change in properties such as resistance, capacitance, inductance, volume or color in response to a target molecule of interest such as THC. For example, polymers can go through a tertiary reorganization based on temperature that results in a physical property change (viscosity, size, etc). The selectivity of the device is enhanced by incorporating highly specific binding receptors (e.g. antibodies) into the sensor material which receptors bind to specific targets (e.g. peptide epitopes). The binding of the target molecule to the receptor causes sensor material to undergo a property change which causes the signaling component (e.g. a piezoresistor) to create a detectable signal (e.g. change in resistance, capacitance or inductance) thereby indicating the present of the target substance.

In one embodiment, MEMS sensors can be used. Another embodiment can use change in color of the sensor or change in size (swelling) as detected by a camera with image processing to detect the presence of the substance. Other implementation can include resonators including a tuning fork (where the polymer spans the two forks) or a resonator attached to a substrate to which the polymer is attached. Putting a resonator into a hydrogel provides a very sensitive detection means as the internal viscosity of the hydrogel changes. The embodiment also specifically includes a resonator coated with phase change polymer wherein the device (coated resonator) is placed into an environment such as water having an analyte. Such a system performs well for either a hydrogel or a crystalline or glassy polymer maintained at a static temperature or subjected to a controlled temperature ramp.

A schematic of an integrated gas sensor system is shown in FIG. 1. Gas sensor 100 includes two metal electrodes 20 and gas sensitive layer 30. Various electrode structures, such as interdigitated electrode structures, can be used. Electrodes can be built using the top metal layers of standard CMOS process with CMOS circuitry implemented on substrate.

Interface circuits to measure the change in resistance, capacitance or impedance (real and imaginary components) can be implemented on chip. The integrated microsystem can also include radiofrequency transceiver for wireless connectivity. Control logic and memory can be used to store and process readings. The sensor or system can be powered by a battery or use energy harvesting techniques to collect energy from ambient energy sources, such as, for example, solar energy, thermal energy, or radiofrequency energy, or combinations thereof. The metal organic framework materials can be used as the main functional element (gas sensitive layer 30) or as an auxiliary element. If used as the main functional element, the metal organic framework material is deposited or grown onto sensing electrodes 20 and the electric properties of the material is monitored upon exposure to the gas analyte. The electrical property being monitored can be the impedance (real and imaginary components), resistance, or capacitance of the sensing layer.

Most metal organic framework materials are dielectrics. In one case, the capacitance can be the property being monitored. The change in capacitance can be due to a change in dielectric constant or swelling of the gas sensitive layer upon exposure to a specific gas. The deposition of metal organic framework materials as the gas sensitive material on the electrodes and the configuration of the gas sensor can take many forms. The bond pad etch can be used to remove any passivation layer and deposit the gas sensitive material 30 directly on top of metal electrodes 20. Interfacial layer can be grown or deposited between electrodes 20 and gas sensitive material 30 as a passivation layer or adhesion layer. In this case only the capacitance of gas sensitive material 30 can be monitored. Electrodes 20 can also have multilayer structure to increase the surface area, hence the total capacitance and the percentage of electric fields lines passing through gas sensitive material 30.

To be used as an auxiliary element of the gas sensor, metal organic framework material can act as a filter to enhance the selectivity of the gas sensor. A metal organic framework filter film can be deposited on top of gas sensitive material 30, which can be another metal organic framework material or a different material.

For measuring the small variations of resistance, a Wheatstone bridge can be used to convert the change in resistance into voltage. For capacitance to voltage conversion, continuous time or discrete time circuits can be used. Using fully differential circuits can yield improved performance. The voltage signal can be further digitized by on-chip analog-to-digital conversion circuitry. Using differential signal arrangement can yield improvement in performance. To sense different cannabis materials, several dies (each with a different metal organic framework material) can be integrated in a single package. Alternatively, different metal organic framework materials can be grown on the same die where each one is selective to a different gas. Multiple metal organic framework materials can be also used to sense the same gas, but the units can have different physical parameters to have different dynamic range, sensitivity, etc., and sensor fusion techniques are used to produce an overall enhanced response. For example, metal organic framework materials can be different and have difference size or thickness. In order to compensate for the effect of humidity, in addition to the gas sensitive material, another material can be used to sense humidity. A temperature sensor can be also integrated in order to compensate for the effect of temperature on the sensor response. There are a few available methods to form metal organic framework materials on electrodes. Conditions are selected to achieve adhesion and stability for the gas sensor application. Metal organic framework materials can be formed from solvothermal mother solutions. In this approach, the substrate with sensing electrodes can be simply immersed into the solution during metal organic framework formation. Another method can be microwave-induced thermal deposition. To make a gas sensor with multiple layers of materials, liquid phase epitaxy (LPE) can be used. The substrate with sensing electrodes can be immersed into solutions of the reaction partners in a sequential, stepwise fashion to synthesize crystalline metal organic framework materials as thin films. The device can be powered by a battery and/or an energy harvesting device to collect energy from one or more of ambient energy sources, including solar energy, thermal energy, or radio frequency energy.

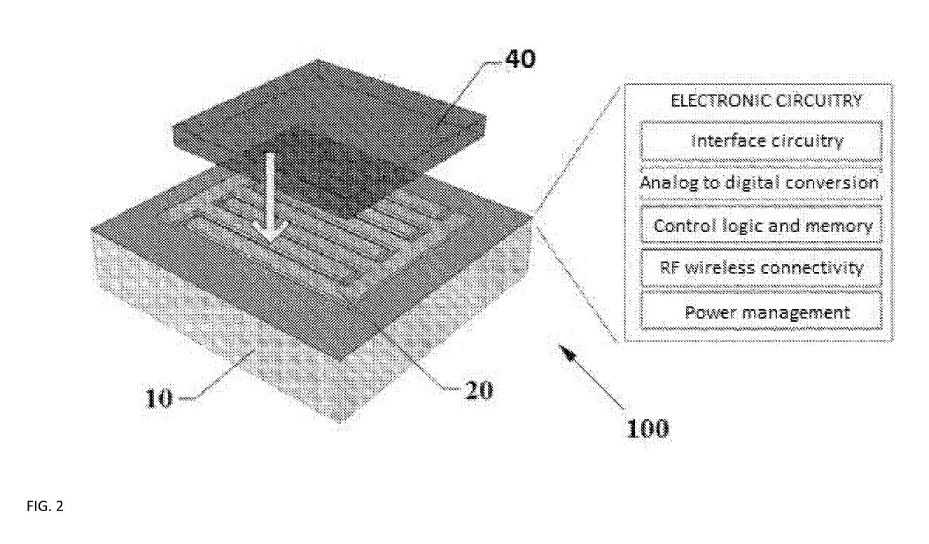

In another aspect, a system can be used to rapidly test urine, saliva or sweat for the presence of Δ-9-tetrahydrocannabinol (THC). In one embodiment shown in FIG. 2, a hydrogel 40 is provided above the electrode 20. The hydrogel 40 contains an antibody that specifically binds Δ-9-tetrahydrocannabinol (THC)—i.e., THC capture antibody. As used herein, “specific” and variations thereof refer to having a differential or a non-general (i.e., non-specific) affinity, to any degree, for a particular target. Also as used herein, the term “antibody” generally refers to a preparation that includes at least one species of immunoglobulin or fragment thereof (e.g., scFv, Fab, F(ab′)2, Fv, or modified forms thereof). Thus, the term “antibody” may include a polyclonal antibody preparation, a monoclonal antibody, a fragment of an immunoglobulin, or any combination thereof. Antibody that specifically binds to THC is commercially available (e.g., Lifespan Biosciences, Inc., Seattle, Wash.; Epitomix Inc., Burlingame, Calif.).

THC (Δ-9-tetrahydrocannabinol), if present in the sample, will bind to the immobilized Δ-9-tetrahydrocannabinol capture antibody with a hydrogel, where it then becomes mobilized and is subject to labeling and detection by components of a reagent solution. The generation of a detectable signal indicates presence of THC in the saliva sample at a concentration of at least a predetermined threshold level, discussed in more detail below. When the control capture area is present, the antibody immobilized in the control capture area immobilizes the saliva component (e.g., amylase) to which it specifically binds, where it is subject to labeling and detection by components of the reagent solution. When present, the generation of a detectable signal in the control capture area signifies that the assay functioned properly so that the absence of a detectable signal in the THC capture area can be interpreted as a negative result. The detectable signal may be generated by one or more components of the reagent solution. Preferably, the detectable signal is electrical in nature (resistance, capacitance, or inductance value change, for example. In some embodiments, the reagent solution can include one or more reagents necessary to generate a fluorescent or colorimetric signal. Such reagent can include a detection antibody that specifically binds to a target captured in the detection zone—e.g., THC captured in the THC capture area or, if a control capture area is present, the saliva component (e.g., amylase) captured in the control capture area.

In addition, the article can be calibrated to generate a detectable signal when a tested sample possesses a predetermined threshold level of THC that reflects acute exposure, but to not generate a detectable signal when a tested sample reflects chronic ingestion. In this way, the articles and methods described herein can assist law enforcement, medical professionals, employers, employees, parents, etc. determine whether a tested sample reflects cannabis use that can impair the function of the subject. In some embodiments, the predetermined threshold level of THC in the saliva can be at least 14.0 ng/mL such as, for example, at least, 14.2 ng/mL, 14.4 ng/mL, 14.6 ng/mL, 14.8 ng/mL, 15.0 ng/mL, 15.2 ng/mL, 15.4 ng/mL, 15.6 ng/mL, 15.8 ng/mL, or at least 16.0 ng/mL.

The assay may be performed on a sample having any suitable volume. In some embodiments, the sample may have a minimum volume of at least 1 μl such as, for example, at least 5 μl, at least 10 μl, at least 15 μl, at least 25 μl, at least 50 μl, at least 75 μl, at least 100 μl, at least 125 μl, or at least 150 μl. The maximum sample volume can be any volume capable of being contained by the device and is, therefore, more a function of preferred design parameters than any technical limitation. In some embodiments, therefore, the sample may have a maximum volume of no more than 5 ml such as, for example, no more than 2 ml, no more than 1 ml, no more than 500 μl, no more than 250 μl, no more than 200 μl, no more than 150 μl, no more than 100 μl, no more than 75 μl, or no more than 50 μl. In some embodiments, the sample volume may be expressed as a range having endpoints defined by any minimum volume listed above and any maximum volume listed above that is greater than the minimum volume.

Next, potential hydrogels for THC detection is detailed. Hydrogels are three dimensional networks of hydrophilic polymers which have been tied together to form water-swellable but water insoluble structures. The term hydrogel is to be applied to hydrophilic polymers in a dry state (xerogel) as well as in a wet state. These hydrogels can be tied together in a number of ways. Firstly, radiation or radical cross-linking of hydrophilic polymers, examples being poly(acrylic acids), poly(methacrylic acids), poly(hydroxyethylmethacrylates), poly(glyceryl methacrylate), poly(vinyl alcohols), poly(ethylene oxides), poly(acrylamides), poly(N-alkylamides), poly(N,N-dimethylaminopropyl-N′-acrylamide), poly(ethylene imines), sodium/potassium poly(acrylates), polysaccharides e.g. xanthates, alginates, guar gum, agarose etc., poly(vinyl pyrrolidone) and cellulose based derivatives. Secondly, chemical cross-linking of hydrophilic polymers and monomers, with appropriate polyfunctional monomers, examples include poly(hydroxyethylmethacrylate) cross-linked with suitable agents, the copolymerisation of hydroxyethylmethacrylate monomer with dimethacrylate ester crosslinking agents, poly(ethylene oxide) based polyurethanes prepared through the reaction of hydroxyl-terminated poly(ethylene glycols) with polyisocyanates or by the reaction with diisocyanates in the presence of polyfunctional monomers such as triols, and cellulose derivates cross-linked with dialdehydes, diepoxides and polybasic acids. Thirdly, incorporation of hydrophilic monomers and polymers into block and graft copolymers, examples being block and graft copolymers of poly(ethylene oxide) with suitable polymers, poly(vinyl pyrrolidone)-co-polystyrene copolymers, polyurethanes and polyurethaneureas and polyurethaneureas based on poly(ethylene oxide), polyurethaneureas and poly(acrylonitrile)-co-poly(acrylic acid) copolymers, and a variety of derivatives of poly(acrylontriles), poly(vinyl alcohols) and poly(acrylic acids). Fourthly molecular complex formation between hydrophilic polymers and other polymers, examples being poly(ethylene oxides) hydrogel complexes with poly(acrylic acids) and poly(methacrylic acids). Lastly, entanglement cross-linking of high molecular weight hydrophilic polymers, examples being hydrogels based on high molecular weight poly(ethylene oxides) admixed with polyfunctional acrylic or vinyl monomers.

It is possible to produce hydrogels which are physically extremely weak when swollen in water so that they flow either under their own weight or under low shear. However, the preferred hydrogels are characterized in that when swollen fully with water they do not flow under their own weight. Preferably they have also significant strength so that they can transmit the osmotic pressure which develops within their structure when they swell in water. A further desirable but not essential feature is that they are tough and not brittle materials in the dry (or xerogel) non-hydrated state; that is xerogel materials which exhibit a glass transition temperature (Tg) well below ambient temperature are preferred. Preferably the Tg is below conceivable use temperatures of the sensor. Many hydrogel materials have high Tg values well above ambient temperatures and may be brittle and weak in use on extreme flexing. They can, however, be utilized as such extreme flexing is rarely encountered.

A preferred group of hydrophilic polymer comprising hydrogels are Poly(ethylene glycols) (PEGs) based hydrogels either crosslinked or made as chain-extended or block copolymers. Such crosslinked copolymers can be made via the reaction of the hydrogel ends of the PEGs with a diisocyanate and a polyol. These are known as polyurethanes and are described for example in UK Patent No. GB 2047093B, UK Patent No. 1506473, European Patent Application Publication No. 0205815 and International Patent Application (PCT) Publication No. WO89/07117. The block copolymers of PEGs can also be made utilising only difunctional units such as, for example, a combination of poly(ethylene glycol), poly(propylene glycol), a diisocyanate and optionally a diamine.

A further preferred group of hydrophilic polymer comprising hydrogels are based on linear chain-extended poly(ethylene oxide) polyurethaneurea hydrogels (UK GB22354620) and a series of linear poly(ethylene oxide)-co-poly(propylene oxide) block copolymer polyurethaneurea hydrogels. These polyurethaneurea (PUU) materials are able to absorb and swell in aqueous media, while retaining their mechanical integrity. The degree to which the polymeric hydrogels will absorb and swell with aqueous solutions is determined by the amount of hydrophilic poly(ethylene oxide) (PEO) incorporated within their structures. The higher the PEO content, the greater the swellability of the hydrogel material. The PUU hydrogels, when swollen, can have equilibrium aqueous media contents ranging from 5-95% by weight at ambient temperature. The hydrogels also exhibit changes in swelling with variations in temperature and may be described as “temperature responsive hydrogels.”

As a result of their linear structure and chemical composition, the PUU hydrogels are soluble in a number of relatively “mild” organic solvents such as methanol, ethanol, propan-2-ol, methyl ethyl ketone, dichloromethane and chloroform. The solubility of the PUU hydrogels means that they can be readily fabricated into films or devices by solvent casting techniques or used in coating applications. The absorption of aqueous media by the PUU hydrogels produces an increase in their physical dimensions and this change can be used to exert a mechanical force or pressure. The speed and extent of the swelling and dimensional response of the PUU hydrogels is determined by their degree of hydrophilicity, governed by the PEO content, their physical dimensions and to the temperature of the system. The poly(ethylene oxide) based PUU hydrogel systems have an inverse swelling response in aqueous media with increasing temperature. A swollen PUU hydrogel will decrease in swelling as the temperature of the system is increased. The decrease in swelling of the PUU hydrogel will result in a contraction of the physical dimensions of the material which can be used to produce a mechanical response. The hydrogels described can be manufactured by various known processes and have the advantage that they are solvent soluble and therefore can be made in a form suitable for coating. They are also thermoplastic and may be extruded into fibres from the melt (with or without plasticizers). The nature of such materials is explained in “Polymer Science and Materials”, Tobolsky, A. V. M. and Mark, H. F., Wiley-Interscience 1971, the content of which is incorporated by reference.

The poly(ethylene oxide) based polyurethaneurea hydrogels can be used in combination with poly(acrylic acids) or poly(methacrylic acids) to produce pH responsive hydrogels through the formation of macromolecular, hydrogen-bonded association complexes between the polyether and the polyacid segments within the hydrogel structures. These materials are soluble in solvent systems and are therefore suitable for the production of pH responsive hydrogel films and coatings. It has been demonstrated that the PUU/polyacid complexed membranes have a low to high swelling response at about pH4.0 in citrate/phosphate buffer systems over the range pH2.2-pH8.0.

The swelling behavior of poly(acrylamide-co-acrylic acid) copolymer gels in response to changes in ionic strength and pH, indicate that swelling responses can also occur in environments of different ionic strengths and at both low and high pH values. At very low pH the poly(acrylamide-co-acrylic acid) gel will deswell to the volume of an unionised gel. As the pH is increased the gel will increase in swelling as the acid groups become ionised until at high pH values, (>pH10), the gel begins to deswell due to the increased concentration of cations within the gel. It has been shown that the PUU/polyacid hydrogels display this type of swelling response at high pH values (pH10-pH12).

A further group of hydrophilic polymers comprising Polymeric microgels have been developed (UK patent GB2090264B), via a solution polymerisation process, comprising crosslinked particles which are capable of forming a sol in the reaction solvent. These crosslinked particles or microgels can be designed to have specific functionalities, reactivities, solubility and size. Basic poly(methylmethacrylate-co-(dimethylamino)ethylmethacrylate) microgels have been developed which, when incorporated into a PUU hydrogel matrix produce pH responsive hydrogel materials which exhibit a change in swelling at about pH6-7. The versatility of the microgel process means that microgels can be prepared which will respond to any chosen external stimuli. For example, microgels incorporating acidic and/or basic groups will respond to changes in pH and/or ionic strength, poly(hydroxyethylmethacrylate-co-dinitrophenol)microgels will respond to the presence of amines, poly(hydroxyethylmethacrylate-co-azobenzoate) microgels will respond to UV radiation and poly(N,N-alkyl substituted acrylamides) based microgels will have a swelling response in relation to the system temperature.

Microgels can be combined in a two component system with a PUU hydrogel matrix, or a carrier or binder to produce responsive hydrogel materials, which swell or deswell i.e. shrink, on exposure to the specific target.

Another class of preferred hydrogels includes acrylamide based polymers, for example polyacrylamide, poly-n-isopropyl acrylamide, poly-n-methyl acrylamide, poly-n,n-dimethyl acrylamide and their methyacrylamide analogs. It is understood that these polymers may be essentially homopolymers or may be co-polymers with another hydrophilic or hydrophobic monomer.

The device is capable of detecting a wide range of target molecules with a high degree of sensitivity and selectivity.

Another aspect of the embodiment is that the sensor material undergoes a dramatic change in volume whereby a hydrogel sheet about 5, 10, 20, 50 or 100 microns thick expands or contracts 0.5% or more, 1% or more, 5% or more, or 10% or more and is detected by the arm capable of detecting movement of in a range of 1 to 1,000 angstroms or more which may include a phase change.

Another aspect of the embodiment is that the sensor material uses a highly specific binding receptor such as a monoclonal antibody or nucleotide sequence which only binds to a single target and not to other molecules which may be closely related to the target.

Another embodiment provides a screen-printed carbon electrode with the hydrogel for the detection of Δ9-THC in undiluted saliva using N-(4-amino-3-methoxyphenyl)-methanesulfonamide mediator. The sensor is optimized for response to Δ-THC in undiluted saliva. Electrochemical oxidation of OX0245 results in oxidation to the diimine, which then reacts with Δ-THC at the 4-position on the phenolic ring, forming an adduct which has two resonance structures, III and IV. The adduct itself can be electrochemically reduced via the diimine of resonance structure IV, and therefore the response to Δ9-THC is observed as an increase in reduction current at the diimine reduction potential, since THC, and therefore also the adduct, are relatively insoluble and readily adsorb onto the electrode, giving an enhanced reduction current in addition to the reduction current arising unreacted mediator II which has diffused to the electrode surface. The sensors consisted of a two electrode system using a carbon working electrode surrounded by a combined Ag/AgCl reference/counter electrode. The mediator and buffer solution are gels formed on the sensor. The hydrogel receives the mediator solution and applied directly onto the sensor. On application of sample, the mediator dissolves and diffuses to the working electrode where it can undergo reaction. The hydrogel/reagent overlayer containing mediator, buffer, salt and surfactant over the electrodes. On applying sample to one end of the membrane, the sample wicked along the overlayer, wetting the reagents and the electrode surfaces. OX0245 has good solubility of at least 1 mg/mL in pH 9.5 buffer, and hence it dissolved off the overlayer rapidly. Initially, the electrochemical procedure has (1) trigger; (2) wait time; (3) galvanostatic oxidation (G) and (4) chronoamperometric reduction (CA). The procedure includes an electrochemical trigger, using the cut-off function within the Nova software of the Autolab instrument. The cut-off consisted of an applied potential of −0.3 V with a time limit of 100 s. The next step of the procedure was triggered when the working electrode current was greater than −100 nA, which typically took 5-10 s after application of sample to the overlayer. The trigger was followed by a brief wait time, to allow the reagents on the overlayer to fully wet up and reach the electrode. During the wait time, the working electrode was at open circuit potential, which typically stabilized at −0.04 V. It was found that more viscous saliva samples took longer to completely wet the overlayer, and a wait time of 20 s was chosen to ensure complete wetting of the overlayer. Galvanostatic oxidation or potentiostatic oxidation can be used, dependent on the viscosity of the saliva sample. Variation in the mediator concentration at the electrode surface will result in a variable amount of oxidized mediator being produced during potentiostatic oxidation, since the rate of oxidation will be dependent directly on mediator concentration as described by the Cottrell equation. Galvanostatic oxidation requires there to be sufficient mediator present to ensure oxidation of only the mediator. Insufficient mediator would result in oxidation of any other oxidizable species present, such as phenolic groups at the carbon electrode surface. Therefore a high mediator loading of 1 mg/mL was used on the overlayer. The magnitude of the shift in potential of the working electrode during the G step gives an indication of whether there is sufficient mediator available. Excessively large potential shifts indicate insufficient mediator.

Yet another aspect of the embodiment is a signaling component which undergoes a change such as a change in resistance, resonant frequency, electrical output, or capacitance in response to very small movements of the arm, or the case of a resonator, to the rheological properties of the materials it is contact with.

Another embodiment uses portable gas chromatography with a MEMS Chemi-Capacitive Detector (Vernier Mini GC Plus Gas Chromatograph) that allows air to be used as a carrier gas. The MEMS chip sensor can be set at either of two levels of sensitivity. Standard sensitivity mode works well for polar compounds such as: ketones, alcohols, and esters. High sensitivity mode works well for compounds such as halogenated alkanes and substituted aromatics, as well as mixtures with one or more compound of low concentration. In one embodiment, the GC sample clean-up and derivatization of target analytes, and call for a capillary GC column that can produce reliable identification and quantification results. Δ9-carboxy-THC is the primary target in GC/MS confirmation analysis, but other marijuana metabolites present in samples include cannabinol, cannabidiol, 11-hydroxy-Δ9-tetrahydrocannabinol (Δ9-hydroxy-THC), Δ9-tetrahydrocannabinol (Δ-THC), and Δ8-tetrahydrocannabinol (Δ8-THC). Further, a guard column can be used to prevent non-volatile residue in the sample matrix from contaminating the analytical column. MTBSTFA (N-methyl-N-(tert-butyldimethylsilyl)-trifluoroacetamide) can be used to derivatize the target compounds.

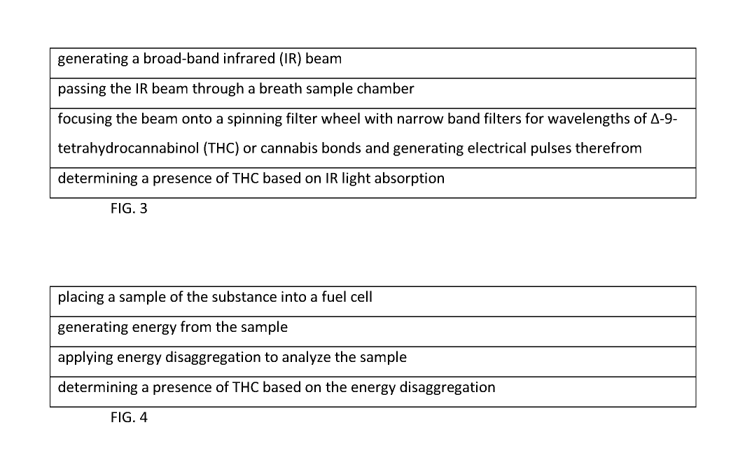

Generally, in some embodiments, the article includes a substrate that includes a detection zone and a fluid collection reservoir. The detection zone generally includes antibody that specifically binds Δ-9-tetrahydrocannabinol. Moreover, the substrate provides fluid communication between the fluid collection reservoir and the detection zone. One embodiment uses infrared (IR) spectroscopy in parallel to detect alcohol impairment, THC impairment, or both. In this system, the person being tested breathes into the device, and part of the breath sample is sent to the THC gas analyzer described above, and part of the breath sample is bubbled through a mixture of sulfuric acid, potassium dichromate, silver nitrate and water. The sulfuric acid removes the alcohol from the air into a liquid solution, and alcohol reacts with potassium dichromate to produce: chromium sulfate potassium sulfate acetic acid water, and during a reaction, the reddish-orange dichromate ion changes color to the green chromium ion when it reacts with the alcohol; the degree of the color change is directly related to the level of alcohol in the expelled air. This embodiment can detect the presence of one or both of alcohol and THC. The changes in vibration include the bending and stretching of various bonds. Each type of bond within a molecule absorbs IR at different wavelengths. So, to identify ethanol in a sample, the wavelengths of the bonds in ethanol (C—O, O—H, C—H, C—C) are detected to measure the absorption of IR light. The absorbed wavelengths help to identify the substance as ethanol, and the amount of IR absorption tells how much THC or ethanol is there. FIGS. 3 and 4 show exemplary processes for IR detection of THC and/or alcohol:

generating a broad-band infrared (IR) beam
passing the IR beam through a breath sample chamber
focusing the beam onto a spinning filter wheel with narrow band filters for
wavelengths of Δ-9-tetrahydrocannabinol (THC) or cannabis bonds and
generating electrical pulses therefrom
determining a presence of THC based on IR light absorption.
placing a sample of the substance into a fuel cell
generating energy from the sample
applying energy disaggregation to analyze the sample
determining a presence of THC based on the energy disaggregation

Another embodiment uses a fuel cell with two platinum electrodes with a porous acid-electrolyte material sandwiched between them. As the exhaled air from the user flows past one side of the fuel cell, the platinum oxidizes THC and/or alcohol in the air to produce acetic acid, protons and electrons. The electrons flow through a wire from the platinum electrode. The wire is connected to an electrical-current meter and to the platinum electrode on the other side. The protons move through the lower portion of the fuel cell and combine with oxygen and the electrons on the other side to form water. The more THC/alcohol that becomes oxidized, the greater the electrical current. A computer measures the electrical current and calculates the alcohol level and the THC level at once. The detection of alcohol level and THC level when both are present can be done using electrical disaggregation (NILM) approach where energy generated by alcohol is detected alone, energy generated by THC is detected alone, and energy generated by both THC and alcohol is detected separately, and each data set is used to train a pattern recongizer. Changes in the voltage and current are measured (i.e. admittance measurement unit), normalized (scaler) and recorded (net change detector unit). A cluster analysis is then performed to identify when different generators are turned on and off.

In another embodiment, a polymer coated on to a waveguide is monitored via an evanescent wave at a selected wavelength for shifts in absorbance induced by the adsorption of THC or its metabolites onto the polymer. Fiber based detection can be used as it localizes chemical recognition at the surface of a waveguide. The sensing rely on the evanescent field of a fiber are absorption and fluorescence. Multimode silica fibers with diameters much larger than the free-space wavelength of light is used. Subwavelength silica fibers in a Mach-Zehnder-type interferometer can be used to detect index changes caused by THC molecules interacting with the surface of the fibers and these sensing configurations offer high sensitivity, fast cycling times, and reversibility as fiber sensors that operate as on/off detectors. In other embodiments with multiple analytical modes for chemical identification, single-crystalline SnO2 nanoribbons can be used the passive optical components in the devices, because their high refractive indices (n≥2 for the wavelengths used here) and high aspect ratios allow efficient waveguiding over extended distances in a microfluidic flow cell.

As delta-9-tetrahydrocannabinol (THC) is the main source of the pharmacological effect, population statistics are used to enhance the accuracy of estimated pharmacokinetic parameters and to develop a population pharmacokinetic model for THC in occasional cannabis smokers and in heavy smokers. Population pharmacokinetic analysis can be performed using a non-linear mixed effects model, alond with demographic and biological data as covariates. The population pharmacokinetic model can describe the quantitative relationship between administration of inhaled doses of THC and the observed plasma concentrations after smoking cannabis. A learning machine is used. Machine learning tasks are typically classified into three broad categories, depending on the nature of the learning “signal” or “feedback” available to a learning system. For example:

Supervised learning: The computer is presented with example inputs and their desired outputs, given by a “teacher”, and the goal is to learn a general rule that maps inputs to outputs.

Unsupervised learning: No labels are given to the learning algorithm, leaving it on its own to find structure in its input. Unsupervised learning can be a goal in itself (discovering hidden patterns in data) or a means towards an end (feature learning).

Reinforcement learning: A computer program interacts with a dynamic environment in which it must perform a certain goal (such as driving a vehicle or playing a game against an opponent[6]:3). The program is provided feedback in terms of rewards and punishments as it navigates its problem space.

Between supervised and unsupervised learning is semi-supervised learning, where the teacher gives an incomplete training signal: a training set with some (often many) of the target outputs missing. Transduction is a special case of this principle where the entire set of problem instances is known at learning time, except that part of the targets are missing.

A support vector machine is a classifier that divides its input space into two regions, separated by a linear boundary. Here, it has learned to distinguish black and white circles.

Among other categories of machine learning problems, learning to learn learns its own inductive bias based on previous experience. Developmental learning, elaborated for robot learning, generates its own sequences (also called curriculum) of learning situations to cumulatively acquire repertoires of novel skills through autonomous self-exploration and social interaction with human teachers and using guidance mechanisms such as active learning, maturation, motor synergies, and imitation.

Tasks can be categorized into deep learning (the application of artificial neural networks to learning tasks that contain more than one hidden layer) and shallow learning (tasks with a single hidden layer).

In classification, inputs are divided into two or more classes, and the learner must produce a model that assigns unseen inputs to one or more (multi-label classification) of these classes. This is typically tackled in a supervised way. In regression, also a supervised problem, the outputs are continuous rather than discrete. In clustering, a set of inputs is to be divided into groups. Unlike in classification, the groups are not known beforehand, making this typically an unsupervised task. Density estimation finds the distribution of inputs in some space. Dimensionality reduction simplifies inputs by mapping them into a lower-dimensional space.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

Unless otherwise specified, the particular examples, materials, amounts, and procedures described herein are exemplary and are to be interpreted broadly in accordance with the scope and spirit of the embodiment as set forth herein.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

The complete disclosure of all patents, patent applications, and publications cited herein are incorporated by reference in their entirety. In the event that any inconsistency exists between this disclosure of this application and the disclosure(s) of any document incorporated herein by reference, the disclosure of this application shall govern. The foregoing detailed description and examples have been given for clarity of understanding only. No unnecessary limitations are to be understood therefrom. The embodiment is not limited to the exact details shown and described, for variations obvious to one skilled in the art will be included within the embodiment defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the embodiment are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for detecting a substance in a subject, comprising:
providing a polymer responsive to a change in property when exposed to Δ-9-tetrahydrocannabinol (THC) or cannabis;
contacting the polymer with a breath from the subject;
immobilizing a Δ-9-tetrahydrocannabinol capture antibody;
binding the THC to the antibody; and
detecting a presence of the substance in the subject by detecting a change in polymer property and comparing the change to predetermined values for THC or cannabis.

2. The method of claim 1, wherein the material comprises an antibody for Δ-9-tetrahydrocannabinol (THC) and an antibody for alcohol to detect the presence of THC, alcohol, or both.

3. The method of claim 1, wherein the polymer is a cross-linked hydrogel.

4. The method of claim 1, wherein said detecting comprises monitoring the polymer for a change in polymer property to detect a body fluid osmolality, wherein the polymer property comprises resistance, inductance, capacitance, volume or color.

5. The method of claim 4, wherein the saliva osmolarity is in the range of 50-200 mOsm.

6. The method of claim 1, wherein said polymer is comprised of one or more monomers selected from the group consisting of an acrylamide or a hydroxy alkyl acrylate.

7. The method of claim 1, wherein said polymer is selected from the group consisting of poly(acrylic acids), poly(methacrylic acids), poly(acrylamides), poly(vinyl alcohols), and poly(ethylene oxides).

8. The method of claim 1, wherein said polymer comprises a monomer selected from the group consisting of hydroxyethylacrylate, acrylamide, methacrylamide, n,n-dimethylacrylamide, hydroxypropyl acrylate, and n-isopropylamide.

9. The method of claim 1, comprising: forming a first gas sensitive region on a substrate by placing a first gas sensitive metal organic framework material proximate to a first pair of electrodes, wherein the electronic property of the first gas sensitive metal organic framework material monitored by a sensor to detect one or more of impedance, resistance, capacitance, volume or color.

10. The method of claim 1, comprising detecting a presence of alcohol, THC, or both.

11. A method for monitoring a substance in a subject, comprising:
generating a broad-band infrared (IR) beam;
passing the IR beam through a breath sample chamber;
focusing the beam onto a spinning filter wheel with narrow band filters for wavelengths of Δ-9-tetrahydrocannabinol (THC) or cannabis bonds and generating electrical pulses therefrom;
determining a presence of THC based on IR light absorption.

12. The method of claim 11, comprising:
focusing the beam onto a second spinning filter wheel with narrow band filters for wavelengths of alcohol bonds and generating electrical pulses therefrom;
determining a presence of THC, alcohol, or both based on IR light absorption.

13. The method of claim 11, comprising:
focusing the beam onto the spinning filter wheel with additional narrow band filters for wavelengths of alcohol bonds and generating electrical pulses therefrom;
determining a presence of THC, alcohol, or both based on IR light absorption.

14. A method for monitoring a presence of a substance in a subject, comprising:
placing a sample of the substance into a fuel cell;
generating energy from the sample in the fuel cell;
applying energy disaggregation to analyze the generated energy from the sample;
determining a presence of THC based on the energy disaggregation.

15. The method of claim 14, wherein the energy disaggregation detects the presence of THC, alcohol, or both.

16. The method of claim 14, wherein the energy disaggregation comprises noninvasive generator monitoring.

17. The method of claims 1, 11 or 14, comprising applying a learning machine to population statistics and adjusting the predetermined values used to detect the substance.

18. The method of claim 1, wherein a polymer coated on to a waveguide is monitored via an evanescent wave at a selected wavelength for shifts in absorbance induced by the adsorption of THC or its metabolites onto the polymer.
